(19) United States
(12) Patent Application Publication
(10) Pub. No.: US 2012/0090619 A1
(43) Pub. Date: Apr. 19, 2012

(54) INFLATABLE RESPIRATORY CUFF WITH PRESSURE RELIEF VALVE

(76) Inventor: Walter Levine, Delray Beach, FL (US)

(21) Appl. No.: 13/089,974

(22) Filed: Apr. 19, 2011

Related U.S. Application Data
(60) Provisional application No. 61/326,042, filed on Apr. 20, 2010.

(51) Int. Cl. A61M 16/04

(52) U.S. Cl. 128/207.15

(57) ABSTRACT

A respiratory cuff pressure regulator system is provided for use with a breathing tube equipped with an inflatable cuff, a tubule extending from the cuff and having a distal end communicating with an inside of the cuff, a proximal end having an inlet for inflating the cuff. The system includes a sealed housing enclosing an inflatable member and in fluid communication with the tubule, the sealed housing having an air inlet bore in fluid communication with the tubule inlet and with the interior of the inflatable member. A valve in the inlet bore is normally closed and opens for inflation of the inflatable member and ultimately the respiratory cuff. An equalizing biasing member in the sealed housing exerts a bias against the inflatable member. A pressure relief valve is provided in fluid communication with the tubule and is preset to open upon pressure in the tubule exceeding a predetermined amount.

### RELATED APPLICATION

This application is a Non-Provisional of, and claims 35 USC 119(e) priority from U.S. Provisional Application Ser. No. 61/326,042 filed Apr. 20, 2010.

### BACKGROUND

The present invention relates generally to tubes insertable into bodily cavities for medical purposes and having inflatable cuffs for retaining such tubes in place in the body, and more specifically to systems for controlling internal cuff pressure.

U.S. Pat. No. 4,501,273, incorporated by reference herein, discloses an apparatus for inflating and maintaining pressure on a cuff used on a breathing tube such as an endotracheal tube, tracheostomy tube or the like. A sealed valve chamber includes an inflatable member inflated by a syringe used to also inflate the cuff. The inflatable member is in fluid communication with the cuff, and expands and contracts in response to pressure changes in the cuff. Externally of the inflatable member and within the sealed valve chamber, springs are provided to exert a biasing force against the inflation of the inflatable member. The springs are assisted in biasing the inflatable member by fluid pressure in the endotracheal tube, with which the sealed chamber is in fluid communication. Thus, as the gas under pressure exits into the interior of the trachea, and increases, the pressure within the cuff will correspondingly or proportionately also increase to prevent collapsing of the cuff, thereby preventing leakage or breakdown of the seal between the inflated cuff and the internal wall of the trachea.

Since the development of the device disclosed in U.S. Pat. No. 4,501,273, it has been found that when a patient being artificially ventilated using an endotracheal tube, tracheostomy tube or the like equipped with the device is also anesthetized, the plastic used to make the cuff absorbs Nitrous Oxide (N₂O) gas used in anesthesia, which increases pressure in the cuff. This increased pressure exerts an excessive force upon the inflatable member, increasing pressure within the cuff. While the desired internal cuff pressure ranges from 18-27 cmH₂0 of water pressure (13.23-19.86 mmHg), in some cases, cuff pressures as high as 125 cmH₂0 (91.94 mmHg) have been recorded.

Such high internal cuff pressure irritates the surrounding tracheal tissue. In many cases, these high cuff pressures can cut off oxygen flow to the tissue, causing tissue death. In reaction, the surrounding tissue swells, in some cases severely restricting or even cutting off the airflow in the trachea. Medical technicians monitoring the patient during anesthesia in some cases fail to monitor the internal cuff pressure, such as by only sporadically visually monitoring the above-described device.

### SUMMARY

The above-identified drawback of the prior art is met by providing a respiratory cuff pressure regulator system associated with a breathing tube, such as an endotracheal tube, wherein the system features a pressure relief valve which is in fluid communication with both an inflatable member and an interior of the cuff. The pressure relief valve is set to open at a maximum pressure, for example in the range of 25-27 cmH₂0 (18.39-19.86 mmHg). In the event the internal cuff pressure rises beyond the desired value, the pressure relief valve opens, releasing excess pressure to ambient and maintaining the appropriate internal cuff pressure. In this manner, the unwanted tissue damage, death and/or swelling is prevented, and the patient's respiratory health is maintained during the use of the endotracheal tube. In a further embodiment, the pressure relief valve is provided with an auxiliary biasing device to regulate the above-identified pressure relief, so that pressure is released slowly in a seeping manner and not all at once. This relatively slow release of pressure prevents the endotracheal cuff from deflating upon the opening of the pressure relief valve.

More specifically, a respiratory cuff pressure regulator system is provided for use with a breathing tube equipped with an inflatable cuff, a tubule extending from the cuff and having a distal end communicating with an inside of the cuff, a proximal end having an inlet for inflating the cuff. The system includes a sealed housing enclosing an inflatable member and in fluid communication with the tubule, the sealed housing having an air inlet bore in fluid communication with the tubule inlet and with the interior of the inflatable member. A valve in the inlet bore is normally closed and opens for inflation of the inflatable member and ultimately the respiratory cuff. An equalizing biasing member in the sealed housing exerts a bias against the inflatable member. As such, air inflation pressures within the inflatable member and the cuff are maintained substantially constant even though their respective inflation volumes vary. A pressure relief valve is provided in fluid communication with the tubule and is preset to open upon pressure in the tubule exceeding a predetermined amount.

In another embodiment, a pressure relief valve is provided for use with a respiratory cuff pressure regulator system for use with a breathing tube having distal and proximal ends, a normally collapsed flexible tubular cuff encircling the tube near the distal end and having opposite ends sealed thereto, a tubule extending from the cuff and having a distal end communicating with an inside of the cuff, a proximal end of the tubule having an inlet for pressurized air for inflating the cuff, the regulator system including a sealed housing enclosing an inflatable member having an interior in fluid communication with the proximal end of the tubule, the tubule passing through an inlet in the housing for engaging the inflatable member, the sealed housing having an air inlet bore in fluid communication with the tubule inlet and with the interior of the inflatable member, a valve in the inlet bore normally closing the inlet and upon exposure to pressurized air, the inlet bore opening for inflation of the inflatable member and ultimately the respiratory cuff, at least one equalizing biasing member disposed in the sealed housing between the inflatable member and the sealed housing for exerting a substantially constant bias against the inflatable member in the sealed housing whereby air inflation pressures within the inflatable member and the cuff are maintained substantially constant even though their respective inflation volumes vary. The pressure relief valve includes a valve housing defining a chamber in fluid communication with the tubule and enclosing a stem biased to a closed position by a biasing member, the biasing member being preset to open upon pressure in the tubule exceeding a predetermined amount, and having a damping member for preventing rapid depressurization of said tubule.

### BRIEF DESCRIPTION OF THE DRAWINGS

FIG. 1 is a side view of a prior art pressure regulator for endotracheal cuffs;

FIG. 2 is a cross-section taken along the lines II-II of FIG. 1 and in the direction generally indicated;

FIG. 3 is an exploded side view in partial section of the present cuff pressure regulator;

FIG. 4 is vertical cross-section of the assembled cuff pressure regulator;

FIG. 5 is an enlarged vertical section of the pressure relief valve employed in the present cuff pressure regulator;

FIG. 6 is a vertical cross-section of an assembled alternate embodiment of the present pressure relief valve;

FIG. 7 is a vertical cross-section of the assembled cuff pressure regulator equipped with the pressure relief valve of FIG. 6; and

FIG. 8 is an exploded rear perspective view of the pressure regulator of FIG. 6.

### DETAILED DESCRIPTION

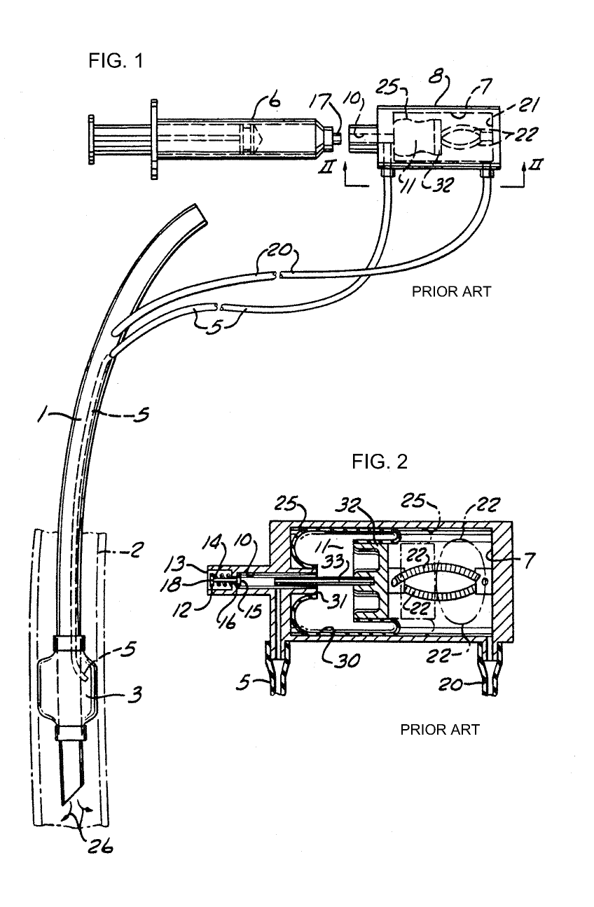

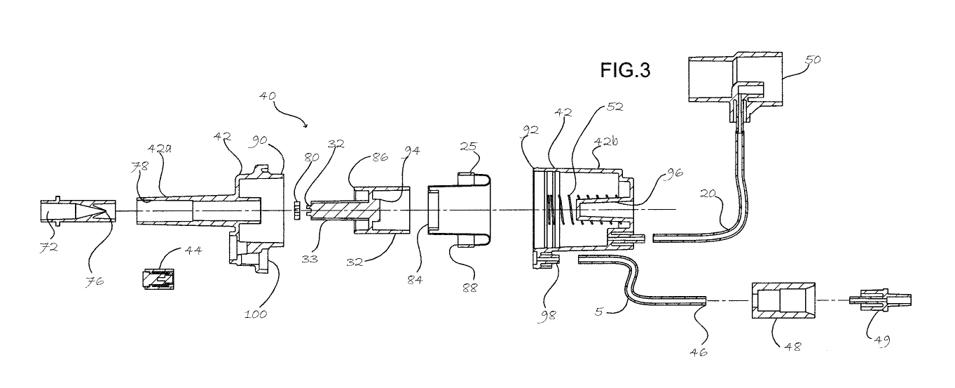

Referring to FIGS. 1 and 2, the prior art system disclosed in U.S. Pat. No. 4,501,273 (incorporated by reference herein) is illustrated. A flexible breathing tube 1 is adapted to be inserted into a trachea 2 has an outer or proximal end 1a for connection to a breathing apparatus such as a respirator (not shown) that will periodically force air into the lungs through the tube. The tube 1 is encircled near its inner or distal end 1b by a flexible cuff 3, which is attached to the tube and forms an air chamber around it. The cuff 3 normally is more or less collapsed against the tube 1 so that it and the tube can be inserted in the trachea 2.

The surgeon, other medical practitioner or caregiver selects a tube diameter based on his estimate of the diameter of the trachea 2. To inflate the cuff 3 so as to form a seal with the wall of the trachea 2, a much smaller tube or tubule 5 is connected with the inside of the cuff and extends out along the inside of the larger tube 1 and then exists as illustrated in FIG. 1.

The proximal inlet of the tubule 5 communicates with an interior of an inflatable member or diaphragm 25, which in turn is expandable within a chamber 7 of a sealed housing member 8. The sealed housing 8 is also provided with an air inlet bore 10 communicating with the tubule inlet of the tubule 5 and with an interior 11 of the inflatable member 25.

A push valve 12 (see FIG. 2) is provided within the inlet bore 10 and normally closes the inlet 13 thereof. Normally closed valve 12 includes a simple valve structure normally biased to a closed position by a compression spring 14 such that a valve head 15 normally closes off and seats against an annular valve seat 16. When it is desired to insert air under pressure into inlet 13, a tip 17 of a conventional syringe 6 is inserted into the inlet 13 and thereby depresses a contact head 18 of the valve 12 inwardly against the compression bias of the spring 14 to unseat the valve head 15 from the annular seat 16 and permit gases under pressure to flow through bore 10 from the plunger action of the syringe 6 to inflate both the cuff 3 and the inflatable member 25. In view of the inflatable member 25 and the cuff 3 having their interiors communicating with each other, they inflate with equal pressures. The result is, as the inflatable member 25 is inflated via the syringe 6 through the check valve 12, so also the inflatable cuff 3 inflates with the same interior pressure to seal off the inner wall of the trachea 2.

The inflatable member 25 is also enclosed within the sealed chamber 7 of the sealed housing 8 for expansion and contraction therein, and a second tubule 20 communicates the sealed chamber 7 with the interior of the endotracheal tube 1. To maintain a constant pressure at a preset or predetermined value within the inflatable member 25 and the cuff 3, a constant pressure structure is provided or disposed in the sealed housing 8 between the inflatable member 25 and an interior wall 21 of the sealed housing in the form of an equalizing biasing member 22, preferably taking the form of two constant force compression coil springs. The springs 22 are constantly bowed outwardly as indicated in their natural state, thereby urging the inflatable member 25 into compression. These springs 22 exert a substantially constant pressure against the inflatable member 25 as it expands from its contracted position as indicated in full lines in FIG. 2 to its expanded position as indicated by the dashed lines in FIG. 2. Thus, the springs 22 exert a substantially constant force against the inflatable member 25 as it expands or contracts throughout a predetermined limited range as indicated to maintain preset air inflation pressures within the inflatable member 25 and the cuff 3 substantially constant even though their respective inflation volumes may vary.

As previously noted, the interior 7 of the sealed housing 8 communicates with the interior of the endotracheal tube 1 via the tubule 20. Thus, as gases under pressure are injected into the interior of tube 1 from the proximal end, and pass through the tube and exit at the distal end thereof in the trachea 2 as indicated by the arrows 26, this gas pressure is also passed from the interior of tube 1 through the tubule 20 into the interior 7 of sealed housing 8, such that these gas pressures also act against inflatable member 25, tending to collapse it under pressure and thereby increase the gas pressure sealed within the interior 11, and to correspondingly increase the gas pressure contained within inflated cuff 3. Thus, as the gas under pressure exits into the interior of trachea 2 as indicated at 26 and increases, the pressure within the inflated cuff 3 will correspondingly or proportionately also increase to prevent collapsing of the cuff 3, thereby preventing leakage or breakdown of the seal between the inflated cuff 3 and the internal wall of the trachea 2. It is thus intended that the cuff 3 does not over-inflate to cause injury, and on the other hand, it is also intended that inflated cuff 3 will always be inflated with sufficient air under pressure to prevent leakage.

The inflatable member 25 is preferably a sleeve-like flexible section of material 30 which is annularly sealed by gluing or other means to a hub 31, and annularly sealed at its other end by similar means to the annular periphery of a piston 32. The piston 32 is provided with a center piston guide rod 33 which is very loosely received within the bore 10 to permit reasonable guided axial movement of the piston without restricting the passage of gases under pressure through the bore 10 into the interior 11 of the inflatable member 25 and into the inlet or interior of tubule 5.

Referring now to FIGS. 3-5, the present system is disclosed, generally designated 40. Components shared with the system of FIGS. 1 and 2 are designated with identical reference numbers. In general, while components have been redesigned to reflect current manufacturing techniques, the present system 40 takes the place of the sealed housing member 8 described above in relation to FIGS. 1 and 2, and has the same general principles of operation in the way the cuff 3 is inflated, and how pressure is equalized between the cuff 3 and internal pressure within the trachea 2.

A main feature of the system 40 is that a sealed housing 42, made up of portions 42a and 42b which are joined together with adhesive, ultrasonic welding or the like, has replaced the sealed housing 8. The sealed housing 42 has been modified to accommodate a pressure relief valve 44 in fluid communication with the inflatable member 25. While the valve 44 is shown mounted to the housing 42, as being integrally molded or joined with adhesive or the like, it is contemplated that it can be optionally located externally of the housing as long as it is in fluid communication with the inflatable member 25 and the tubule 5, which also inflates the cuff 3. A free end 46 of the tubule 5 is provided with a fitting 48 having an internal nipple 49. The free tubule end 46 matingly and slidingly engages the nipple 49, which is configured for releasably frictionally engaging a luer type fitting (not shown) on the cuff 3 for achieving fluid communication between the chamber 7 and specifically between the inflatable member 25, the pressure relief valve 44 and the cuff.

In addition, the tube 20, which connects the housing 8 and the sealed chamber 7 with the endotracheal tube 1, is optionally provided with an adapter 50 for facilitating connection with the endotracheal tube, or with a respiratory therapy faceplate (not shown) as is known in the art. Another feature of the system 40 is that the equalizing biasing member springs 22 have been replaced with a single coil spring 52.

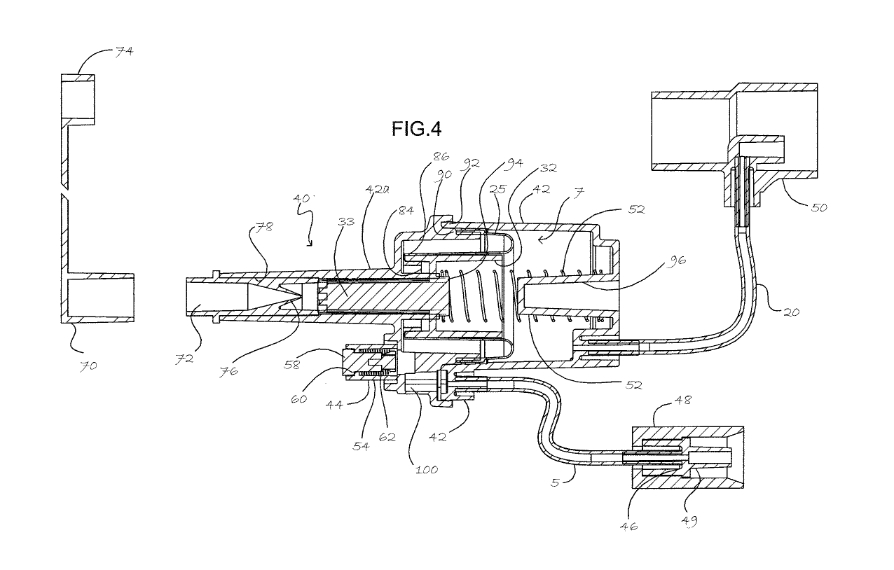

Referring now to FIGS. 4 and 5, the valve 44 is shown described in greater detail. A valve housing 54 is secured to the sealed housing 42, such as being integrally molded therewith, or secured by chemical adhesives or ultrasonic welding as is known in the art. The valve housing 54 encloses a valve stem 56 having a larger diameter head 58 sealingly engaging an annular stop ring 60, which is part of the valve housing. Reciprocating within the valve housing 54, the valve stem 56 is subject to a biasing force exerted by a biasing member 62, in the preferred embodiment taking the form of a coiled spring or the like. Opposite the head 58, the valve stem 56 has an end plug 64, also with a larger diameter end 66 that captures the biasing member 62 within a chamber 68 in the valve housing 54. As seen in FIG. 5, stop ring 60 projects into the chamber 68, and the end plug 64 is matingly secured to the valve stem 56, such as by chemical adhesive, ultrasonic welding, threading or the like.

Upon an increase in pressure in the cuff 3, there will be a corresponding increase in pressure in the tubule 5, which is in communication with the valve housing 54. The pressure relief valve 44, more specifically the biasing member 62, is preset to open at a designated pressure, such as 25-27 cmH₂0 (18.39-19.86 mmHg). The specific target pressure may vary to suit the application. Thus, once the pressure increases beyond the preset amount, the biasing force of the biasing member 62 is overcome, allowing the stem head 58 to move out of sealing engagement with the stop ring 60, or to the left as shown in FIGS. 4 and 5. In this manner, excess pressure is released to ambient, and the cuff pressure is still maintained at sufficient levels to provide a snug engagement with the interior of the bodily orifice, such as the trachea 2.

Referring now to FIG. 4, another feature of the present system 40 is that a tethered cap 70 is provided for use on an open end of a syringe inlet 72. The cap 70 is configured to sealingly engage the inlet 72, and preferably also has a valve cap 74, which is configured to engage the valve housing 54 at the end adjacent the head 58 to prevent escape of air or pressure when desired. It will be appreciated that the valve cap 74 may be secured to the sealed housing 42 separately from the cap 70 (FIG. 3) to prevent loss and/or to facilitate user accessability as is known in the respiratory therapy art. Also, in the system 40, it will be appreciated that the inlet push valve 12 is replaced by a molded flap-type check valve 76, however other types of check valves are contemplated.

Referring again to FIGS. 3 and 4, additional details of the construction of the system 40 will be described. Beginning at the left end of FIG. 3, the syringe inlet 72 is fastened within an inlet bore 78 of the sealed housing portion 42a. In the preferred embodiment, the housing portion 42a is made of a transparent or translucent material, for reasons described below. Reciprocating within the inlet bore 78 is the center guide rod 33 of the piston 32. In the system 40, a colored indicator 80 is placed on a tip 82 of the guide rod 33 and is visible through the inlet bore 78. In some applications, the indicator 80 is fluorescent, as are the respective ends of the inlet bore 78 for facilitating monitoring of cuff pressure by medical technicians, especially at night. Thus, a technician can visually monitor the status of the pressure within the housing 42 as well as the cuff.

At an opposite end of the guide rod 33 from the tip 82, a free end 84 of the flexible member 25 is secured to the piston 32, as by being folded around a piston edge 86. An opposite end 88 of the flexible member 25 is secured between adjacent edges 90, 92 of the sealed housing portions 42a, 42b as they are joined together. The coil spring 52 is seated at one end upon an end 94 of the guide rod 33 as it projects through an interior of the piston 32. At its opposite end, the spring 52 is seated upon a lug 96 projecting internally of the housing 42b.

It will also be seen that the tubule 5 is in communication with the pressure relief valve 44 through engagement of the tubule with a housing nipple 98 in fluid communication with a portion 100 of the interior chamber 7 with which the pressure relief valve 44 is also in fluid communication. Thus, the syringe inlet 72, the space defined by the flexible member 25, the pressure relief valve 44 and the tubule 5 are all in fluid communication.

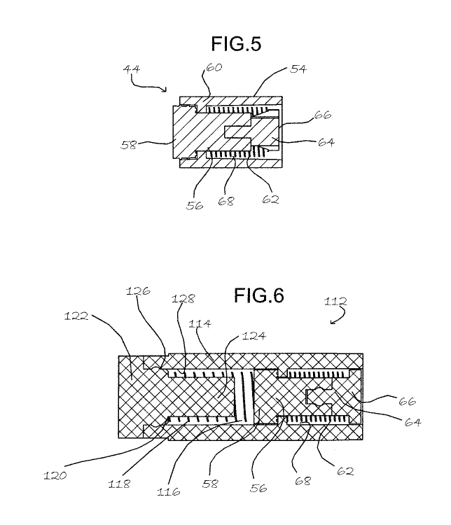

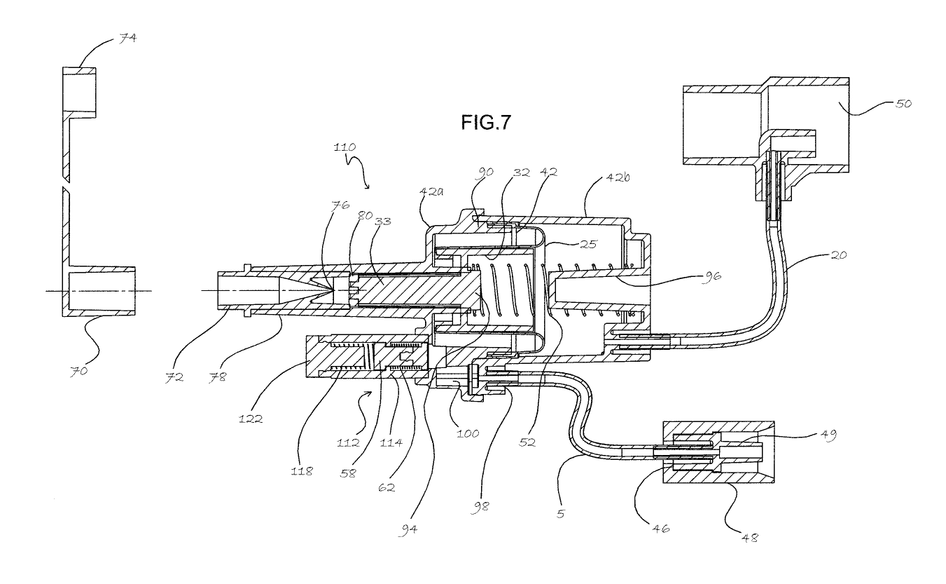

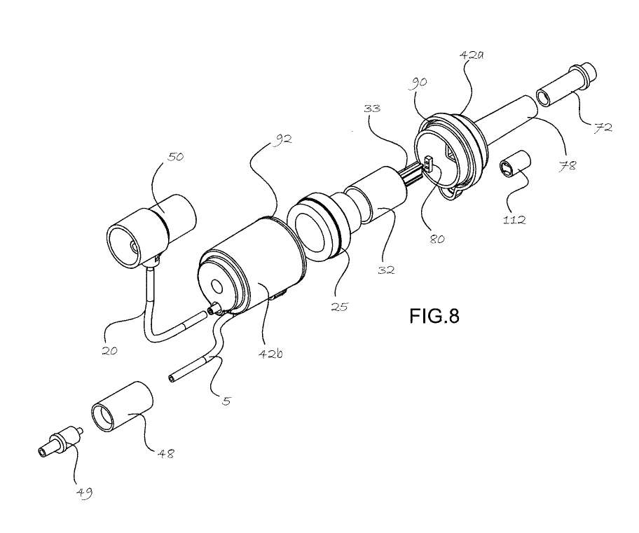

Referring now to FIGS. 6-8, another embodiment of the cuff pressure regulating system 40 is generally designated 110. Components shared with the system 40 are designated with identical reference numbers. A main distinction between the systems 40 and 110 is that the pressure relief valve 44 has been replaced with a pressure relief valve 112. Included within a valve housing 114 are the components of the valve 44, designated with their original reference numbers. In addition, the valve housing 114 extends axially from the stem head 58 and defines an inner chamber 116 slidably accommodating the stem head.

A damping member 118, preferably a coiled spring, engages the stem head 58 at one end, and at an opposite end engages a shoulder 120 of an endcap 122. A lug 124 of the endcap 122 extends toward the stem head 58 for locating the biasing member 118 in place. The endcap 122 is held in the housing 114 through a friction fit between a radially extending annular rib 126 which engages an inner wall 128 of the housing. The damping member 118 exerts a biasing force against the stem head 58 in the same direction as the spring 62, however the damping member 118 acts on an opposite end of the stem head 58. The action of the damping member 118 exerts a biasing force against the stem 58 to prevent rapid depressurization upon increased pressure in the tubule. In other words, the damping member 118 retards the opening of the relief valve 44 and thus permits pressure in the tubule 5 and the cuff 3 to be relieved gradually, or in a seeping fashion, instead of a wholesale dumping of pressure if the valve 44 opens quickly, which may tend to deflate the cuff 3. While other settings are contemplated depending on the application, the damping member 118 is set to release upon pressure exceeding 28 cmH₂0, or slightly greater pressure than the setting of the biasing member 62. This setting is intended to allow the desired seeping release of pressure and maintain cuff inflation.

Thus, while the systems 40, 110 monitor cuff pressure, the valves 44, 112 are set to open upon pressure in the tubule 5 and the cuff 3 exceeding a predetermined amount. In the event a technician desires to keep a patient's alveoli open and as such increases ventilator pressure through the adapter 50, there is a potential for excessive pressure to build within the chamber 7 and act on the flexible member 25, which ultimately can increase pressure in the cuff 3. However, the present pressure relief valves 40, 110 prevent cuff pressure from exceeding a predetermined amount. Also, any increase in cuff pressure through the permeation of nitrous oxide into the cuff will be released through the present valves, maintaining cuff inflation while preventing tracheal tissue damage.

While a particular embodiment of the present inflatable respiratory cuff with pressure relief valve has been shown and described, it will be appreciated by those skilled in the art that changes and modifications may be made thereto without departing from the invention in its broader aspects and as set forth in the following claims.

What is claimed:

1. A respiratory cuff pressure regulator system for use with a breathing tube, an inflatable tubular cuff, a tubule extending from the cuff and having a distal end communicating with an inside of the cuff, a proximal end having an inlet for pressurized air for inflating the cuff, said regulator system comprising:
a sealed housing enclosing an inflatable member having an interior in fluid communication with the tubule, said sealed housing having an air inlet bore in fluid communication with the tubule inlet and with said interior of said inflatable member;
a valve in said inlet bore is normally closed and opens for inflation of said inflatable member and ultimately the respiratory cuff;
at least one equalizing biasing member disposed in said sealed housing between said inflatable member and said sealed housing exerts a bias against said inflatable member in said sealed housing whereby air inflation pressures within said inflatable member and said cuff are maintained substantially constant even though their respective inflation volumes vary; and
a pressure relief valve in fluid communication with said tubule and preset to open upon pressure in said tubule exceeding a predetermined amount.

2. The system of claim 1, wherein said pressure relief valve includes a valve housing enclosing a stem biased to a closed position by a biasing member.

3. The system of claim 2, wherein said valve housing defines an internal chamber and includes an inwardly projecting stop engaged by said stem in said closed position.

4. The system of claim 2, wherein said valve housing includes a damping member exerting a biasing force against said stem to prevent rapid depressurization upon increased pressure in said tubule.

5. The system of claim 1, wherein said pressure relief valve is provided with a damping member exerting a biasing force on said valve for preventing rapid pressure release.

6. The system of claim 5, wherein said pressure relief valve includes a valve housing enclosing a stem biased to a closed position by a biasing member, said damping member constructed and arranged for exerting a biasing force on said stem in the same direction as said biasing member, but on an opposite side of said stem.

7. The system of claim 2, wherein said valve housing is secured to said sealed housing.

8. The system of claim 1, wherein said equalizing biasing member is a coiled spring.

9. The system of claim 1, further including a second tubule communicating said interior within said sealed housing with an interior of said breathing tube, said second tubule connected to a luer fitting.

10. The system of claim 1 wherein said tubule has a free end with a connector fitting for enhancing a positive engagement with the cuff.

11. The system of claim 1 wherein said sealed housing is provided with a syringe inlet having a tethered cap, and a valve cap configured for engaging said valve housing for preventing unwanted escape of air.

12. The system of claim 1 further including a visual indicator of the status of the cuff pressure.

13. A pressure relief valve for use with a respiratory cuff pressure regulator system for use with a breathing tube having a normally collapsed flexible tubular cuff encircling the tube, a tubule extending from the cuff and having a distal end communicating with an inside of the cuff, a proximal end of the tubule having an inlet for pressurized air for inflating the cuff, the regulator system including a sealed housing enclosing an inflatable member having an interior in fluid communication with the tubule, the tubule passing through an inlet in the housing for engaging the inflatable member, the sealed housing having an air inlet bore in fluid communication with the tubule inlet and with the interior of the inflatable member, a valve in the inlet bore normally closing the inlet and upon exposure to pressurized air, the inlet bore opening for inflation of the inflatable member and ultimately the respiratory cuff, at least one equalizing biasing member disposed in the sealed housing between the inflatable member and the sealed housing for exerting a substantially constant bias against the inflatable member in the sealed housing whereby air inflation pressures within the inflatable member and the cuff are maintained substantially constant even though their respective inflation volumes vary, said pressure relief valve comprising:
a valve housing defining a chamber in fluid communication with the tubule and enclosing a stem biased to a closed position by a biasing member, said biasing member being preset to open upon pressure in the tubule exceeding a predetermined amount and having a damping member for preventing rapid depressurization of said tubule.

14. The system of claim 13, wherein said valve housing includes an inwardly projecting stop engaged by said stem in said closed position.

15. The system of claim 13, wherein said damping member is constructed and arranged for exerting a biasing force against said stem to prevent rapid depressurization upon increased pressure in said tubule.

16. The system of claim 15, wherein said damping member is constructed and arranged for exerting a biasing force on said stem in the same direction as said biasing member, but on an opposite side of said stem.
